(19) United States
(12) Patent Application Publication
(10) Pub. No.: US 2007/0020211 A1
(43) Pub. Date: Jan. 25, 2007

(54) Betaine with Calcium and/or Strontium Antiperspirants

(75) Inventors: Zijun Li, Westfield, NJ (US); Mark Rerek, Scotch Plains, NJ (US)

Correspondence Address: ARTHUR J. PLANTAMURA;GENERAL CHEMICAL PERFORMANCE PRODUCTS LLC., 90 EAST HALSEY ROAD, PARSIPPANY, NJ, 07054, US

(73) Assignee: REHEIS, INC., Berkeley Heights, NJ (US)

(21) Appl. No.: 11/161,117

(22) Filed: Jul. 22, 2005

(51) Int. Cl. A61K 8/26

(52) U.S. Cl. 424/68

(57) ABSTRACT

Aluminum and aluminum-zirconium antiperspirant compositions comprising basic aluminum chlorides that have a particular molecular size distribution defined by having an SEC-HPLC Band III/II ratio of at least 0.5, having SEC-HPLC Band III plus Band II area of at least 70% of the total area and having SEC-HPLC Band I content no more than 5% and containing betaine (trimethylglycine), calcium and/or strontium are disclosed. Also disclosed are the methods of making these compositions and the use thereof in consumer acceptable antiperspirant vehicles such as aerosols, gels, roll-on, sticks and soft solids.

This invention relates to novel basic aluminum chlorides and basic aluminum chloride/zirconyl hydroxy chloride antiperspirant compositions that have a particular molecular size distribution in the presence of betaine (trimethylglycine) in combination with a calcium and/or strontium compound, to processes for the preparation of the antiperspirant compositions and to consumer acceptable antiperspirant vehicles such as aerosols, gels, roll-on, sticks and soft solids containing such antiperspirant compositions.

### BACKGROUND OF THE INVENTION

Basic aluminum halides, particularly chlorides, and their use as effective antiperspirant compounds are well known. Basic aluminum halides are complex structures made up of mixtures of polymeric and monomeric species of various sizes and molecular structures, depending upon their Al:Halide ratio, together with varying amounts of bound or coordinated water. The basic aluminum compounds are generally represented by the empirical formula:
Al₂(OH)_{(6-X)}Yₓ·nH₂O
wherein Y is Cl, Br or I and 0<x<6 and n is about 0.8 to 4. It should be understood that the above formula is simplified because it is intended to include basic aluminum halides containing coordinated or bound molecules of water as well as basic aluminum halide polymer complexes and mixtures of the above.

The invention also contemplates aqueous aluminum zirconium antiperspirant actives comprising basic aluminum chlorides of the formula
Al₂(OH)₍₆₋ₐ₎Clₐ
where a is a number from about 1.2 to 2.0, from about 5% to 40% by weight having an SEC-HPLC Band III to Band II area ratio of at least 0.5 and a zirconium compound of the formula
ZrO(OH)_{b}CI_{c}
from about 1 5% to 40% by weight wherein b is a numerical number from 0 to 1 and c is at least 1.

It is a challenge to economically prepare enhanced efficacy aluminum and aluminum-zirconium antiperspirant actives at high concentrations of aluminum. Efficacy enhancement is often achieved by changing the molecular weight distribution of the basic aluminum compounds from a larger to a smaller molecular weight distribution. These differences in molecular weight distribution are often expressed by reference to the various aluminum peaks that can be identified when the salt is analyzed by size exclusion chromatography (SEC) typically run by high performance liquid chromatography (HPLC). A suitable chromatographic technique is capable of resolving the major molecular weight segments in BAC into at least four distinct peaks labeled Band I (or peak 1 & peak 2), Band II (peak 3), Band III (peak 4) and Band IV (peak 5). The retention time at which these peaks appear, their resolutions and their respective peak areas, are the function of the column (or columns) and mobile phase used. In general, enhanced efficacy salts have been described as having either enhanced HPLC Band III or peak 4 content or Band III (peak 4) to Band II (peak 3) ratios of at least 0.5. It is important to note that generally, Bands I, II, III and IV of one system correspond respectively to peaks 1 & 2, 3, 4 and 5 of the other system, as described above.

In accordance with the invention we have discovered that superior enhanced efficacy salts should have a size exclusion chromatography SEC-HPLC Band III to Band II area ratio of at least 0.5. At least 70% of the total SEC-HPLC band content should be contained within Band II and Band III, preferably 80%. These enhanced salts have a Band III content of at least 20%, and preferably at least 30%, of the total aluminum contained in all the peaks as measured by peak area. In contrast, conventional non-enhanced antiperspirant salts have Band III content of the range of about 10% and Band III to Band II area ratio from about 0.10 to 0.2 or less. No more than 5% of the SEC-HPLC band content should be in Band I which is significantly lower Band I content than in conventional, non-activated antiperspirant salts. Band IV content should be about 30%, preferably about 20%.

Activation of aluminum antiperspirant actives is normally achieved through dilution of a concentrated basic aluminum chloride (BAC) solution, particularly aluminum chlorohydrate (ACH) solution, followed by heating at elevated temperatures. This process is described in U.K. Patent Application GB 2,048,229 A of Fitzgerald which discloses that diluting ACH solution with water to achieve concentration of at least 5% by weight, preferably 7.5% and more preferably 10% by weight or more, and aging the solution at a temperature above 50° C. and below 100° C. results in the formation of an optimal group of complexes defined as Al^{c}′ and usually present in amounts from about 10% to 30% by weight in the resulting ACH solution. Results obtained according to that reference comprised concentrations between 10% and 25% (page 2, line 3 of that reference). However, the relatively dilute ACH solutions described in GB 2,048,229 A are outside the practical scope of the present invention.

U.S. Pat. No. 4,359,456 of Gosling, et al., discloses that basic aluminum halides obtained by conventional methods can be further broken down from high molecular weight polymers by diluting concentrated solutions thereof to lower aqueous concentrations (aluminum concentration of 10% to 35% by weight) and heating at a temperature of 50° C. to 140° C. for a period of time sufficient to provide a Band III percent aluminum value of at least 20%. No disclosure is contained therein concerning the distribution of, and of any significance attributable to, aluminum species in Bands other than Band Ill. Notable also in that patent disclosure of the significance of the differing process parameters that comprise the present invention.

A method similar to that of U.S. Pat. No. 4,359,456 is described in U.S. Pat. No. 4,775,528 of Callahan, et al., for obtaining enhanced aluminum with zirconium antiperspirant actives. Aluminum chlorohydrate solution is activated by diluting to about 2% to 20% by weight until the ratio of the height of the peak 4 corresponding to k_{d}=0.7, i.e., Band III, to that of peak 3 corresponding to k_{d}=0.5, i.e., Band II , is at least 2:1 and at least 80%, and preferably at least 90%, of the total aluminum is present within the peaks corresponding to peaks 4 and 3 (Bands III and II) (Column 2, lines 60-61). The resulting product contains lower molecular weight polymers to increase efficacy, but suffers by having a wider polydispersity. The antiperspirant composition comprises zirconyl hydroxy chloride solution and an amino acid to provide an atomic ratio of Al:Zr from 6:1 to 1:1. The zirconyl hydroxy chloride solution containing an amino acid may be added before, during or after the heat treatment to obtain the combined enhanced aluminum/zirconium antiperspirant active. There is no range or specification delineated for aluminum species in peaks 1 and 2 and peak 5. Peak 4 should have at least 53% of aluminum species to meet the requirements (a) of peak 4 to peak 3 area ratio of at least 2:1 and (b) sum of peaks 4 and 3 areas to be 80% of the total aluminum present within all the peaks.

U.S. Pat. No. 4,818,512 of Markarian discloses a process for preparing enhanced efficacy aluminum chlorohydrate product containing from about 77% to about 85% of an aluminum chlorohydrate molecular species having a k_{d} value of equal to about 0.4 (Band III species) comprising heating an aqueous solution of about 5% aluminum chlorohydrate at a temperature of from about 60° C. to about 132° C. for a period of from about 30 minutes to about three months to produce a reaction product and spray drying the reaction product. In this reference, the effect of the concentration of aluminum chlorohydrates (a series of ⅚ basic aluminum chlorides were prepared ranging in concentration from 5% to 25% by weight) solution, the time of heating and the temperature of heating on the distribution of various molecular species of aluminum are described. The results disclosed in U.S. Pat. No. 4,818,512 demonstrate that at higher concentrations (25% or higher) the percent of lower molecular Band III and Band IV species obtained is very low (less than 20% and 5% respectively) despite aging at an elevated temperature for more than 3 months. Such compositions and process parameters of U.S. Pat. No. 4,818,512 are clearly outside the range of the improved compositions contemplated by the present invention.

U.S. Pat. No. 4,859,446 of Abrutyn discloses a process for preparing an enhanced antiperspirant which is characterized by size exclusion chromatograph corresponding to Band III of the standard BAC solution and a Band III percent aluminum value of at least 40%. That process comprises reacting an aluminum compound of the formula: AlₙXₘ, wherein X is Cl, Br, F, I, SO₄ and NO₂, n is 1 or 2 and m is 1 or 3, with aluminum metal in an aqueous medium at a temperature between 50° C. and 195° C. until a ratio of aluminum to anion of 0.50-2.5:1 is obtained and recovering the resultant product without elevated temperature aging. The product composition differs as characterized by HPLC as well as the applied process differs from the present invention.

U.S. Pat. No. 4,871,525 of Giovanniello discloses a process which comprises heating in water, at a temperature from about 50° C. to about 100° C., aluminum metal preferably in the form of pellets or powder, with an HX halogen-containing compound wherein X is chlorine, bromine or iodine. The amount of water used is such as to have concentration of the polymer in percent by weight in the range from about 8% to about 35%, preferably from about 15% to about 25%, and more preferably from about 17% to about 22% by weight. The reaction temperatures are preferably in the range from about 95° C. to about 100° C. and should not be elevated so as to create reflux conditions. 100% of the aluminum containing polymers are found in Bands II, III and IV and Band III contains at least 20% of the total aluminum polymers. The resulting product is described as having narrow polydispersity when the batch concentration falls within 17%-22% and metal to chloride atomic ratio does not exceed 2.00:1. The product of the '525 patent differs from that of the U.S. Pat. Nos. 4,359,456 and 4,775,528 patents in that the '525 patent claims zero aluminum species in Band I. It is noteworthy that in contrast to the present invention, none of these three patents discloses, teaches or suggests the preparation of effective compositions at ACH concentrations above 35%. Neither do they disclose the provision of stable solutions at any concentration.

U.S. Pat. No. 4,900,534 of Inward discloses a process for the direct preparation of aluminum zirconium halohydrates of enhanced efficacy having size-exclusion chromatograph of which the Band III proportion is at least 20%. The process parameters and product characterization disclosed in that patent, however, differ significantly from those of the present invention.

U.S. Pat. No. 4,944,933 of Inward discloses a process for the manufacture of basic aluminum chloride solutions with 7.5% to 13% by weight, having an aluminum chloride molar ratio in the range of 1.7 to 2.2:1 and which has Band III fraction of at least 20% and drying the final BAC solution to give hydrated powder having Band III fraction of at least 20%. The patent lacks any disclosure or teaching of a process for obtaining the desired high Band III content in concentrated BAC solutions as does the present invention.

U.S. Pat. No. 5,356,609 of Giovanniello teaches a modification of the direct production of activated aluminum chloride actives through reacting AlCl₃.6H₂O with aluminum metal at 50° C.-100° C. affording a solution of 8%-25% by weight of the desired product. The product has 100% of the aluminum content in Bands II, III, and IV with at least 25% in Band III. U.S. Pat. No. 5,358,694 of Giovanniello teaches a similar method wherein HCl is used in place of AlCl₃.6H₂O. Although these processes avoid the initial production of a non-enhanced BAC solution, both methods result in solutions of lower total solids and have less Band III activated aluminum species in their higher solids examples than the requirement contemplated by the present invention.

U.S. Pat. Nos. 5,202,115, 5,595,729, and 5,626,827 of Barr, et al., disclose antiperspirant active compositions comprising basic aluminum material having the empirical formula Al₂(OH)₆₋ₐXₐ where 0.5≦a≦5 and X is a univalent complex oxo anion of nitrogen or a univalent complex oxo anion of a halogen (for example, NO₃⁻, ClO₃⁻, ClO₄⁻ and IO₄⁻). The products are characterized by chromatography peaks corresponding to peak 3 and peak 4 of the size exclusion HPLC chromatogram with a peak 4 (Band III) relative area of at least 25% and peak 3 (Band II) relative area of less than 60%, the sum of the relative peak 3 and peak 4 (Band II and Band III) area being at least 50% and peak 1 less than 10% (chromatographic peaks eluting at shorter retention times than peak 3 corresponding to peaks 1 and 2). The products are particularly directed to basic aluminum antiperspirant materials containing a univalent complex oxo anion of nitrogen or halogen and specifically directed selectively away from chloride. Accordingly, the compositions of these patents differ from those contemplated for the present invention. It should also be noted that these materials are regarded as not FDA Antiperspirant Monograph approved actives and, therefore, would be unacceptable for use as antiperspirant compositions in the United States.

It is to be noted that the antiperspirant composition of the above references, once produced by heat treatment followed by cooling to room temperature, the activated, lower molecular weight solutions described in these references must be dried to powders quickly due to the rapid loss of activation gained through the heat treatment. The reversibility of the heat activation process prevents these prior art activated products from being used in gels or emulsion roll-ons as the actives must remain in aqueous solution for these types of products.

Calcium and strontium salts have been reported to stabilize HPLC Band III/II ratio of aluminum or aluminum-zirconium solutions in the presence of an amino acid such as glycine. For example, U.S. Pat. No. 6,042,816 of Shen describes a method of stabilizing an aqueous solution of enhanced efficacy aluminum-zirconium antiperspirant salt, containing an effective amount of a water soluble calcium salt and an effective amount of a water soluble amino acid, against rapid degradation of HPLC peak 4 (Band III) to peak 3 (Band II) ratio of the salt. However, high concentration aluminum salt solutions with high peak 4/3 ratio stabilized by calcium in the presence of amino acid such as glycine are not stable, i.e., the solutions gel quickly at RT. Therefore, the challenge (in solution retention) remains to both activate and maintain, i.e., stabilize, the activation in higher concentration solutions of aluminum based antiperspirant actives. High concentrations of the antiperspirant active is desirable to provide formulation efficiency in antiperspirant products.

U.S. Patent Publication No. 2004/0091436 A1 of Li, et al., discloses aluminum and aluminum-zirconium antiperspirants of enhanced efficacy containing strontium and an amino acid such as glycine with stable HPLC Band III/II ratios. Here, again, such high aluminum concentration solutions, as described in U.S. 2004/0091436 A1, are prone to gelling upon standing.

It should be noted that it is a challenge to achieve activation of aluminum antiperspirant compositions of greater chloride content, namely aluminum sesquichloride. Aluminum sesquichloride compositions encompass aluminum to chloride atomic ratios of 1.8 to 1.2. Activation through enhanced Band III content is more difficult at lower aluminum to chloride ratios. While U.S. Pat. No. 6,902,724 of Parekh describes a composition containing optimal molecular weight material, it is necessary to maintain a metal to chloride ratio of between 1.2 to about 1.5, preferably between 1.3 to 1.4, and a solution solids content maintained within a relatively narrow range of about 30% to about 40% anhydrous weight percent and Size Exclusion Chromatography Test Band with a Band I percent aluminum value of less than 5, a Band II value about 20-60%, a Band III value between about 10% and 35% and a Band IV value between 15 to 50. The present invention provides a solution of improved molecular weight, greater Band III content at higher solids and a composition whose solubility stability is maintained.

Various publications describe aluminum and aluminum-zirconium antiperspirants containing betaine. Betaine, also identified as trimethylglycine, and its derivatives including the monohydrate and hydrochloride solids. Trimethylglycine (betaine) is not an amino acid. Trimethylglycine has a quarternary nitrogen group that cannot act as a hydrogen ion donor or acceptor in place of an amino group. The normal form of the compound is as an internal salt, or zwiterion, of the composition (CH₃)₃N+CH₂COO—. As such, it has very different chemistry from glycine. Several recent publications on betaine are summarized below.

WO 2004/026295A2 relates to the use of zwitterionic compounds and their derivatives as protecting agents of skin, hair and nails. The zwitterionic compounds include betaines. Similar filings include WO 2004/089325 A1, EP 1005853 B1, U.S. 2004/0109833 A1 and U.S. 2004/0198998 A1. These properties of betaine are not part of this invention.

WO 2004/089325A1 describes a glycine-free aluminum and/or zirconium betaine salt, where betaine is used as either the monohydrate or the hydrochloride derivative. While a number of different methods are disclosed in the literature for making antiperspirant/betaine salts, none of the known methods describes a process to activate basic aluminum chloride in the presence of betaine. The invention encompasses a method for the addition of betaine to previously activated aluminum chlorohydrol solutions followed by drying as described in great detail hereinbelow.

### SUMMARY OF THE INVENTION

The present invention resides in the discovery that an aluminum salt solution of particular molecular weight associated with enhanced efficacy having HPLC Band III/II ratio of at least 0.5, preferably at least 0.7 and most preferably at least 0.9 with an SEC-HPLC Band III to Band II area of at least 70% of the total area and an SEC-HPLC Band I content of no more than 5% is obtained through the heat treatment of high solids basic aluminum chlorohydrate (BAC) solution (and including alternatively such BAC solutions in combination with a zirconium compound) in the presence of betaine and a calcium and/or strontium salt.

Accordingly, it is an object of the present invention to provide novel enhanced efficacy stable basic aluminum halide antiperspirant (a term which includes aluminum-zirconium antiperspirant) actives which can be produced economically as either solutions or solids.

It is another object of the present invention to provide basic aluminum halide antiperspirants of this kind with substantially enhanced relative efficacy and to provide methods of forming such materials without the need for manufacturing steps previously thought to be necessary, e.g., heating diluted solutions of already manufactured basic aluminum halides at high temperatures and/or pressure conditions.

It is a further object of the present invention to provide the formation of enhanced efficacy basic aluminum halide solutions that have high concentrations thereby minimizing spray drying cost.

It is yet another object of the present invention to provide antiperspirant compositions having enhanced antiperspirancy and skin friendliness.

It is another object of this invention to provide a process for the preparation and stabilization of concentrated BAC and aluminum zirconium solutions with high HPLC Band III/II ratio with excellent physical and chemical stability that can be used in enhanced efficacy gel and emulsion antiperspirant products.

Specifically, the activated aluminum-calcium/strontium-betaine salts are prepared by (1) heating a BAC solution of about 10% to about 50% by weight having an aluminum to chloride ratio of about 1.2 to about 2.0 in the presence of betaine and a calcium and/or strontium salt at a temperature of at least 40° C. and preferably higher, for a period of time sufficient to yield the desired level of activation; (2) reacting Al metal with AlCl₃ or HCl aqueous solution in the presence of betaine and a calcium and/or strontium salt at higher temperatures. Alternatively, the diluted and heated BAC solutions at concentrations of from about 8% by weight to about 20% by weight may be mixed with betaine and a calcium and/or strontium salt.

The aluminum-zirconium-betaine-calcium/strontium salt solutions of enhanced efficacy can be prepared by blending a zirconium salt solution with an activated aluminum-betaine-calcium/strontium salt solution made above to afford an Al/Zr ratio of about 2 to about 10 and a M/Cl ratio of about 0.9 to about 2.0. Preferably the aluminum-zirconium-betaine-calcium/strontium salt solutions is a tetra or octa salt with M/Cl ratio of about 1 to about 1.4 having a betaine/zirconium ratio of about 1 to about 2, preferably about 1. The activated aluminum and aluminum-zirconium-betaine-calcium/strontium salt solutions can be used as is or to be dried to powders for the use in aerosol, soft solid, cream, roll-on, gel, stick formulations, as appropriate.

### BRIEF DESCRIPTION OF THE DRAWINGS

FIG. 1 is the ICP-SEC-HPLC of an aluminum-betaine-calcium salt solution prepared according to the invention.

FIG. 2 is the ICP-SEC-HPLC of an aluminum-zirconium-octa salt with betaine and calcium salt solution prepared according to the invention.

FIG. 3 is the ²⁷Al NMR of an aluminum-zirconium-octa salt powder with betaine and calcium salt prepared according to the invention.

FIG. 4 is the ²⁷Al NMR of an aluminum-zirconium-octa salt solution with betaine and calcium salt prepared according to the invention.

FIG. 5 is the ICP-SEC-HPLC of an aluminum-zirconium-octa salt solution with betaine and strontium salt prepared according to the invention.

### DETAILED DESCRIPTION OF THE INVENTION

The present invention is directed to the preparation of improved, enhanced efficacy, aluminum and aluminum-zirconium antiperspirant salt compositions comprising betaine and a calcium or strontium salt or a combination of calcium and strontium salts wherein the antiperspirant has an HPLC Band III to II area ratio of at least 0.5. The basic aluminum salts of these compositions have the formula:
Al₂(OH)₆₋ₐXₐ
wherein X is Cl⁻, Br⁻, I⁻or NO₃⁻, a is from about 1 to about 2, with basic aluminum chloride solution the most preferred. It is preferable to have a BAC solution with aluminum to chloride ratio of about 1.2 to about 2.0 and most preferably from about 1.4 to about 1.8 depending on the type of calcium and/or strontium compound used in the activation, i.e., a water soluble salt such as calcium and/or strontium chloride or insoluble, highly alkaline base such as calcium oxide or strontium hydroxide or blends thereof. When the water soluble calcium and/or strontium salt is used, BAC solution with higher Al/Cl ratio such as from about 1.6 to about 1.8 is preferred, which gives maximum activation of the aluminum species, i.e., less Band I, preferably less than 5%, and higher Band III/II ratio of at least 0.7. When strong alkaline calcium and/or strontium compounds are used, BAC solutions with lower Al/Cl ratios such as from about 1.2 to about 1.6 are preferred.

According to Process (1) of the present invention, the BAC solutions are mixed with a water soluble calcium and/or strontium salt, preferably calcium and/or strontium chloride, and betaine then heated from about 40° C. to about reflux until an HPLC Band III/II ratio of at least 0.5 is achieved. This process is most preferred since it quickly forms activated aluminum species that are stable in these solutions. When ACH solution (Al/Cl=1.9-2.1) is used, higher HPLC Band I is formed and the activated species are not stable at higher concentrations. A preferred method for Process (2) involves reacting Al powder with AlCl₃ (or HCl), in the presence of betaine and a strongly alkaline base such as CaO or Sr(OH)₂ or a blend thereof for example. At lower Al/Cl ratios such as about 1.3, an activated aluminum solution is formed directly with an HPLC Band III/II ratio of above 0.5. When betaine and a water soluble calcium and/or strontium salt, for example CaCl₂.2H₂O, are used in Process (2), a longer period of time is required to form the activated aluminum species. A high concentration of HPLC Band I (>10%) is formed when the Al/Cl ratio is close to, or above, 1.91 and the solution readily gels. At Al/Cl ratios of less than 1.4, the solution has an HPLC Band III area of less than 20%, or the HPLC Band III/II ratio is less than 0.5. However, the solution Band III/II ratio becomes greater than 0.5 upon aging.

The aluminum antiperspirant solution of the present invention comprises about 10% to about 50% by weight of basic aluminum chlorohydrate salt, preferably about 30% to about 45% by weight; about 1% to about 15% by weight betaine, preferably about 2% to about 8% by weight and about 0.2% to about 15% by weight Ca and/or Sr compound, and preferably about 0.5% to about 8% by weight Ca and/or Sr compound. Typical water soluble calcium salts include calcium chloride, calcium nitrate, calcium bromide, calcium citrate, calcium formate, calcium acetate, calcium ascorbate, calcium sulfate, calcium lactate and mixtures thereof. Typical insoluble, alkaline calcium bases include calcium carbonate, calcium hydroxide, calcium oxide and mixtures thereof. Preferred water soluble strontium salts include strontium chloride, strontium nitrate, strontium bromide, strontium citrate, strontium formate, strontium acetate, strontium ascorbate, strontium lactate and mixtures thereof. Typical insoluble, alkaline strontium bases include strontium carbonate, strontium hydroxide, strontium oxide and mixtures thereof.

According to the present invention it has been discovered that high concentration of aluminum-betaine-calcium and/or strontium solutions of enhanced efficacy are very stable with respect to both composition (HPLC Band III/IlI ratio) and viscosity. This is in clear contrast to the corresponding solutions containing amino acids, such as glycine, (in place of betaine), which gel rapidly at RT.

It has also been found that the aluminum-betaine-calcium and/or strontium solutions of enhanced efficacy prepared by the process of heat treatment of ACH (Al/Cl=1.9-2.1) solution (such as 46.8% by weight) with CaCl₂ and betaine results in a hazy solution with large Band I. In contrast, clear solutions with HPLC Band I of less than 5% are formed from higher concentration solutions of about 48.4% by weight of BAC with Al/Cl ratio of about 1.75 when heat treated with CaCl₂ and betaine. Moreover, when blending these two solutions with a zirconium solution, the resulting zirconium-BAC-betaine-calcium/strontium solution, as illustrated by Example 3 herein below, has a higher Band III/II ratio than for the zirconium-ACH-betaine-calcium/strontium solution.

The BAC solution can be prepared directly, i.e., reacting aluminum powder with AlCl₃ or HCl aqueous solution at elevated temperature or indirectly, i.e., mixing ACH with AlCl₃ or HCl.

Zirconium compounds useful in the present invention have the formula
ZrO(OH)_{b}X_{2-b}
wherein b is a numerical number from 0 to 1.2, X is Cl⁻, Br⁻, I⁻ or NO₃⁻, with chloride being the most preferred. The zirconium hydroxychloride (ZHC) solution can be prepared by the reaction of basic zirconium carbonate with hydrochloric acid or zirconyl chloride at elevated temperature for a certain period of time. It is desirable to have ZHC solutions with Cl/Zr ratio of at least 1, preferably of at least 1.4, and more preferably of at least 1.8.

The aluminum-zirconium-betaine-calcium/strontium salts are prepared by mixing the activated BAC-betaine-calcium/strontium solutions with a ZHC solution. The resultant solutions comprise from about 15% to about 60% by weight of a zirconium-aluminum-chlorohydrate-betaine salt, preferably about 30% to about 50% by weight, about 0.2% to about 10% by weight of calcium/strontium salt, preferably about 0.5% to about 6% by weight. The aluminum-zirconium salt solutions thus prepared have a M/Cl ratio of about 1 to about 2. Preferably the aluminum-zirconium salts are tetra and octa salts, with M/Cl ratio of about 1 to 1.4 most preferred.

A polyhydric alcohol, such as propylene glycol, may be included in the aluminum or aluminum-zirconium-betaine-calcium/strontium solutions, which has the effect of enhancing the stability of the corresponding solutions, especially with respect to inhibition to gelling.

The aluminum and aluminum-zirconium solutions containing calcium/strontium salts and betaine can be dried to powders by any appropriate means, including freeze-drying and vacuum drying. Spray drying is the most preferred method. Furthermore, the solutions can be dried into different densities and different shapes, such as low and high density spherical particles to suit for the variety applications in the formulations like soft solids and low residue sticks.

The characterization of aluminum and zirconium species in aluminum-zirconium-betaine-calcium/strontium antiperspirant salts are determined by the following methods:

SEC-HPLC

The degree of the polymerization of aluminum complexes is determined by Size Exclusion Chromatography (SEC) operated via a High Performance Liquid Chromatograph (HPLC) instrument. In this technique, the highest molecular weight Al species are eluted first and are designated as Band I or otherwise called peaks 1 and 2. Band II (or peak 3) and Band III (or peak 4) are due to intermediate molecular weight Al complexes. Band IV or peak 5 is due to the lowest molecular weight Al complexes, including monomers and dimers. The relative area of one or more peaks is determined in order to characterize the distribution of polymeric species in aluminum complexes formed. The aluminum and aluminum-zirconium antiperspirant salts containing betaine and calcium/strontium salts according to the present invention have SEC-HPLC Band III to Band II area ratio of at least 0.5, preferably at least 0.7, and most preferably at least 0.9. At least 70% of the total SEC-HPLC band content should be contained within Band II and Band III, preferably 80%. These enhanced salts have a Band III content of at least 20%, and preferably at least 30%, of the total aluminum contained in all the peaks as measured by peak area. No more than 5% of the SEC-HPLC band content should be contained in Band I. Band IV content should be about 30%, preferably about 20%.

A Primesphere C1 column (250×4.6 mm) from Phenomenex is used to obtain a SEC-HPLC Chromatogram. Each sample is dissolved in deionized water to form a 2% by weight Al solution. Each sample is filtered through a 0.45μ filter and chromatographed within 15 minutes using a 0.01N nitric acid solution as the mobile phase at a flow rate of 1 ml/minute.

Nuclear Magnetic Resonance Spectroscopy

²⁷Al Nuclear Magnetic Resonance (NMR) is utilized to identify the structures of different aluminum species in the activated aluminum-zirconium antiperspirant salts of enhanced efficacy. The antiperspirant salt in solution form is measured as is and the powder is dissolved in deuteriated water to form a 10% by weight solution just before the measurement. Data were collected using a Varian Inova 400 instrument at 104.2 MHz.

Inductively Coupled Plasma Spectroscopy (ICP) SEC-HPLC

The present invention employs ICP-SEC-HPLC analysis to characterize the distribution of aluminum, zirconium and calcium/strontium species. The size exclusion column operated by HPLC has been used to separate the aluminum and zirconium polymers by size. The separated species are sent to the ICP instead of a refractive index (RI) detector as in regular HPLC. Prior to the analysis, the column was conditioned by repeated injections of a 10% aluminum-zirconium tetrachlorohydrex glycine solution until the area of Band I is constant.

The invention will be further illustrated by the following Examples. In the Examples, parts are by weight unless otherwise specified.

### EXAMPLE 1

Direct Preparation of Aluminum-Calcium-Betaine Solution and the Comparison with Aluminum-Calcium-Glycine Solution

i. 740 parts of AlCl₃ (32° Be) and 1500 parts of water were mixed and heated in a 4-Liter beaker. 14 parts of CaO (Aldrich, 98% purity) was added and the mixture was heated continuously until a clear solution was formed. Following was the gradual addition of 159 parts of Al powder (Alcoa). Water was added during the entire reaction to maintain the reaction temperature and the volume of the reaction mixture. 115 parts of betaine anhydrous (Arch Chemicals, Inc.) was introduced at the end of the reaction and the final solution was filtered to give a clear colorless aluminum-betaine-calcium salt solution.

ii. A similar reaction was carried out except 75 parts of glycine was added at the end of the reaction. The aluminum-glycine-calcium salt solution was a clear fluid after the filtration and the results are listed as follows,

TABLE I

| Experiment | | | | | | | | |
|---|---|---|---|---|---|---|---|---|
| Number | % Al | % Cl | % Ca | % betaine | % glycine | Fresh | 6 MO/RT | 6 MO/40° C. |
| i | 7.99 | 6.72 | 0.42 | 4.51 | — | 0.9 | 0.8 | 0.75^{b} |
| ii | 7.88 | 6.58 | 0.43 | — | 2.97 | 1.0 | a | — |

a: gelled within a week at RT.;
^{b}viscosity = 5 cps

### EXAMPLE 2

Comparison of Aluminum-Betaine-Calcium and Aluminum-Glycine-Calcium Solutions Made by the Heat Treatment of Basic Aluminum Chloride Solutions

954 parts of BAC solution (12.1% Al, 9.1% Cl, Al/Cl ratio of 1.75), 135 parts of CaCl₂.2H₂O (Fisher, 27.2% Ca) and 78 parts of betaine anhydrous (Arch Chemicals, Inc.) were refluxed for 2 hours. The clear and stable BAC-betaine-calcium salt solution has an HPLC Band III/II ratio of about 1.3 after mixing overnight.

In a similar experiment, 318 parts of the same BAC solution was refluxed with 45 parts of CaCl₂.2H₂O crystals and 17 parts of glycine for 2 hours. A hazy solution was formed, which turned creamy overnight and gelled in less than a week.

### EXAMPLE 3

Comparison of Aluminum-Betaine-Calcium Salt Solutions Made from BAC and ACH Solutions

i. 318 parts of BAC solution (12.1% Al, 9.1% Cl, Al/Cl ratio of 1.75), 35 parts of CaCl₂.2H₂O and 15 parts of betaine were refluxed for 2 hours to form solution 3.

ii. 329 parts of ACH solution (11.7% Al, 8% Cl, Al/Cl ratio of 1.92), 35 parts of CaCl₂2H₂O and 15 parts of glycine were refluxed for 2 hours to form a hazy solution 4; the results are listed below,

TABLE II

| | | | | | HPLC Profile | |
|---|---|---|---|---|---|---|
| Experiment | | | | | % | % |
| Number | % Al | % Ca | % betaine | % glycine | Band I | Band III/II |
| i | 10.46 | 2.6 | 4.08 | 0 | 3.2 | 0.87 |
| ii | 10.16 | 2.5 | 0 | 3.96 | 12.5 | 0.96 |

It has been observed that when a solution of ZrOCl₂ was mixed with the resulting solution from Experiment i, the HPLC Band III/II ratio increased to about 1.1. In contrast, when a solution of ZrOCl₂ was mixed with the resulting solution from Experiment Solutions ii, a hazy solution with a lower HPLC Band III/II ratio of 0.77 was formed, demonstrating that betaine is superior to glycine in these antiperspirant compositions and processes.

### EXAMPLE 4

Aluminum-Betaine-Calcium Solutions Prepared at Different Concentration of Calcium Salt and Betaine

Several aluminum-betaine-calcium solutions were made through the heat treatment of a BAC solution (12.1% Al, 9.1% Cl, Al/Cl ratio of 1.75) with different amounts of calcium chloride dihydrate and betaine.

TABLE III

| Experiment Number | % Al | % Ca | % betaine | HPLC Band III/II* |
|---|---|---|---|---|
| i | 9.90 | 3.15 | 6.7 | 1.30 |
| ii | 10.23 | 3.26 | 3.5 | 1.10 |
| iii | 10.45 | 2.59 | 4.1 | 0.74 |
| iv | 10.60 | 1.87 | 5.5 | 0.90 |

*HPLC were preformed the next day after the preparation.

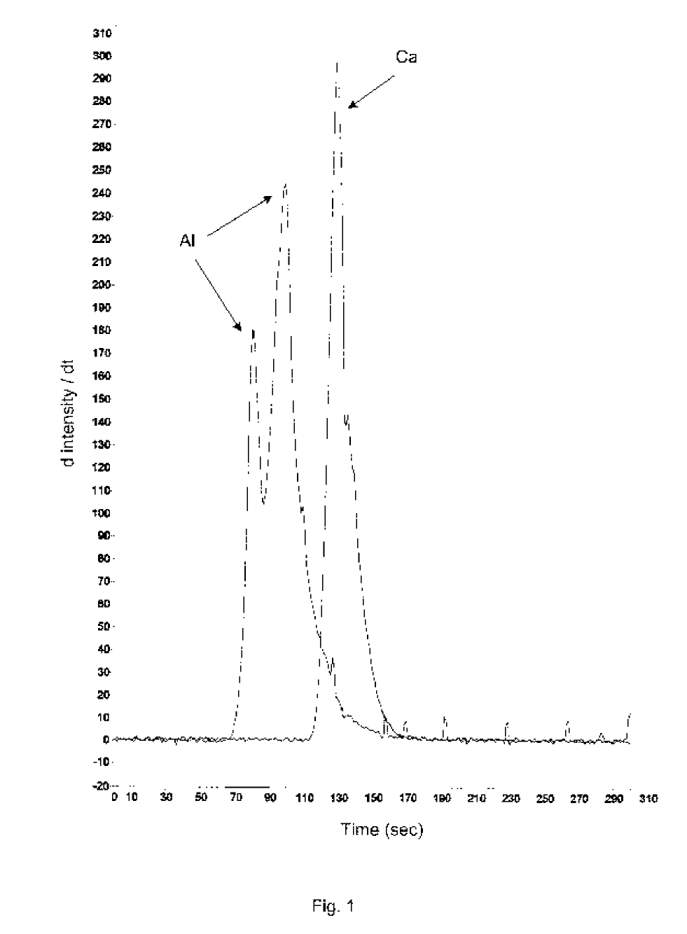

All resulting solutions of Experiments i-iv are stable with respect to both HPLC Band III/II ratio and solution viscosity. The ICP-SEC-HPLC of aluminum-betaine-calcium salt Solution i is shown in FIG. 1. It was subsequently spray dried providing an off-white powder.

### EXAMPLE 5

Aluminum-Calcium-Betaine Solutions Prepared through Direct Process

Aluminum-betaine-calcium solutions were prepared by mixing aqueous AlCl₃ (32° Be) with CaCl₂.2H₂O and betaine. Al powder was gradually added at higher temperature. The mixtures were filtered after the reactions were finished. The results are listed in Table IV.

TABLE IV

| | | | | | HPLC Bands | |
|---|---|---|---|---|---|---|
| | | | | | % | % |
| | | | | | Band I | Band III/II |
| Experiment | | | | | fresh/1 | fresh/1 |
| Number | % Al | Al/Cl | % Ca | % betaine | MO | MO |
| i | 9.19 | 2.05 | 2.05 | 3.08 | 30.2/32.0 | 0.99/1.36 |
| ii | 9.85 | 1.90 | 2.54 | 5.95 | 19.7/16.9 | 1.43/2.04 |
| iii | 9.72 | 1.92 | 2.44 | 2.72 | Gel/1 MO | |
| iv | 9.62 | 1.92 | 2.41 | 3.65 | Gel/4 days | |
| v | 9.48 | 1.72 | 2.37 | 3.27 | 0/0 | 0.55/0.90 |
| vi | 9.68 | 1.72 | 2.54 | 5.50 | 0/0 | 0.77/1.99 |
| vii | 9.39 | 1.32 | 2.33 | 3.16 | 0/0 | 0.36/0.75 |
| viii | 10.06 | 1.30 | 2.56 | 5.72 | 0/0 | 0.26/0.84 |

HPLC data indicate that the aluminum-betaine-calcium solutions made through the direct process (Process 2), at higher Al/Cl ratios such as 1.9 and higher, contain more polymerized aluminum species as indicated by the presence of a greater amount of Band I. The solutions are also less stable, and can gel at aluminum concentrations of above 9%. At Al/Cl ratios of less than 1.4, the HPLC Band III/II ratios of the fresh solutions are usually below 0.5 but become higher on aging. Aluminum-betaine-calcium solutions formed at Al/Cl ratio of about 1.7 by the direct process show no evidence of polymerized aluminum species (0% Band I) and contain activated aluminum species with respect to higher Band III/II ratios of greater than 0.5, especially upon aging. The direct process forms depolymerized and activated aluminum species more slowly compared to those of the solutions made through the indirect heat-activation process. HPLC Band III/II ratio becomes higher with higher concentrations of betaine.

It has been observed that subsequent refluxing deactivates the aluminum-betaine-calcium solutions made by direct process as shown for solutions vi and viii. These were aged for two weeks at room temperature, then refluxed for two hours. The Band III/II ratios dropped from 1.88 to 1.23 in Example vi and from 0.63 to 0.30 in Experiment viii.

### EXAMPLE 6

Preparation of Aluminum-Zirconium-Calcium-Betaine Antiperspirant Salts

i. Through Indirect Process (1)

Several aluminum-zirconium-betaine-calcium octa salt solutions were prepared by mixing aluminum-betaine-calcium solutions made by indirect heat treatment process with ZrOCl₂ solutions and the results are listed in Table V.

TABLE V

| Experiment | | | | | | Band | % Band |
|---|---|---|---|---|---|---|---|
| Number | Al/Zr | M/Cl | % A.S. | % Ca | % betaine | III/II | IV |
| i | 8.35 | 1.37 | 33.3 | 2.54 | 2.70 | 0.6 | 16.5 |
| ii | 8.35 | 1.37 | 33.3 | 2.08 | 3.27 | 1.0 | 24.9 |
| iii | 8.35 | 1.37 | 33.3 | 1.50 | 4.40 | 1.1 | 19.6 |
| iv | 8.35 | 1.37 | 33.3 | 2.54 | 5.43 | 1.6 | 16.7 |

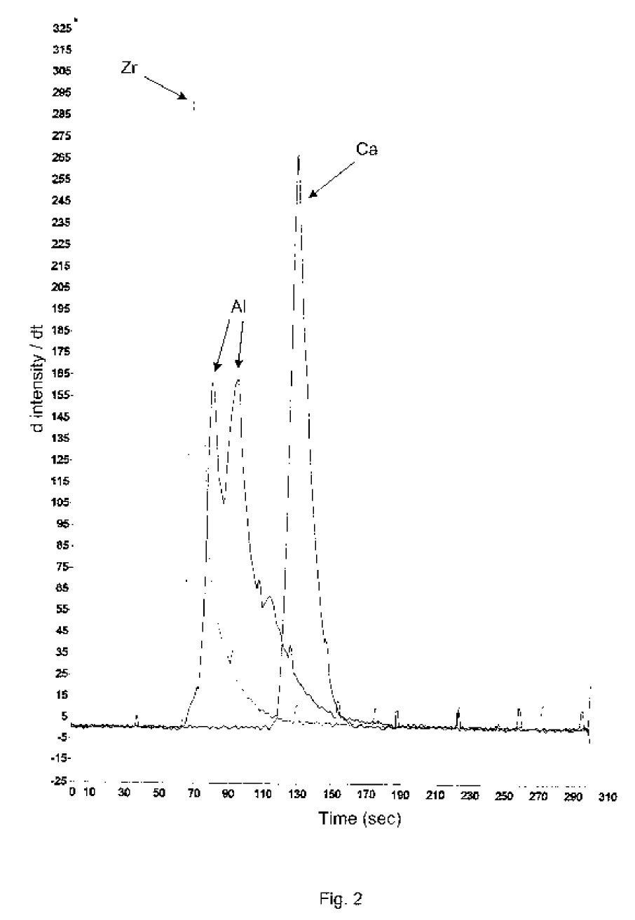

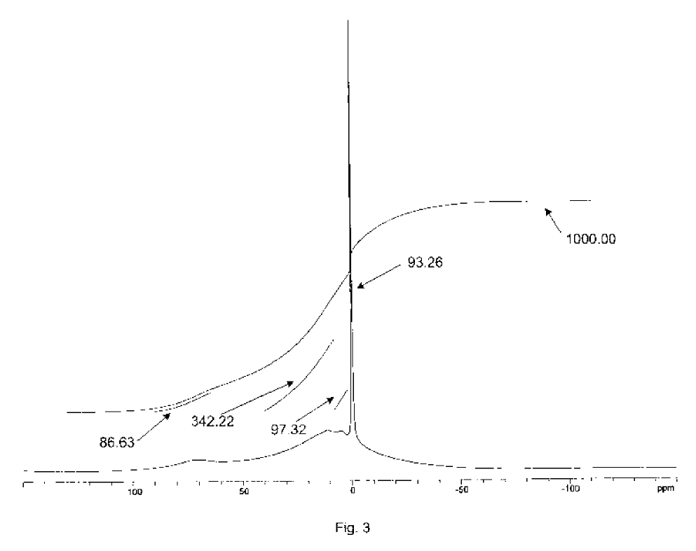

The ICP-SEC-HPLC of solution i is shown in FIG. 2 and the ²⁷Al NMR spectrum of the spray dried powder is shown in FIG. 3.

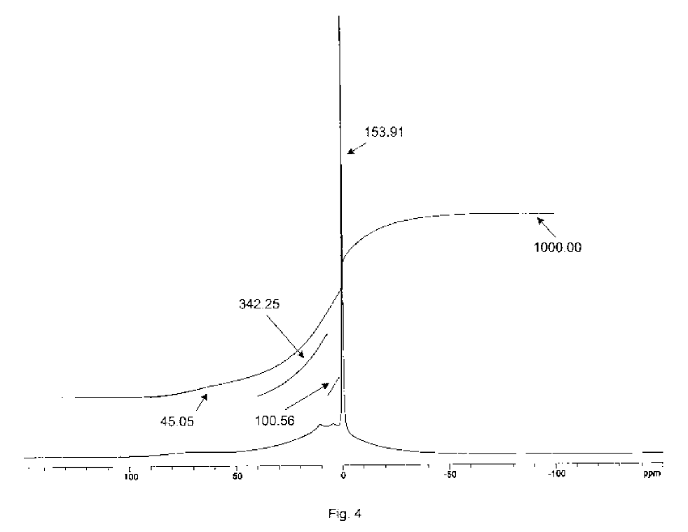

An aluminum-betaine-calcium solution was prepared by refluxing a BAC solution having Al/Cl ratio of 1.38 with CaCl₂.2H₂O and betaine for two hours. ZrOCl₂ solution was added to the above solution to form an aluminum-zirconium-betaine-calcium octa salt solution with Al/Zr ratio of 8.35 and M/Cl ratio of 1.1. The solution has an anhydrous solids content of 34.4% with an HPLC Band I of less than 2% and Band III/II ratio of 1.24. The ²⁷Al NMR is shown in FIG. 4.

ii. Through Direct Process (2)

The aluminum-betaine-calcium solution was prepared through the direct reaction of Al powder with AlCl₃ aqueous solution in the presence of CaO and betaine at elevated temperature. ZrOCl₂ solution was added to make an aluminum-zirconium-betaine-calcium octa salt solution with Al/Zr ratio of 6.8 and M/Cl ratio of 1.1 with anhydrous solid content of 29.1%. Sufficient ZrOCl₂ solution was added to make an aluminum-zirconium-betaine-calcium tetra salt solution with Al/Zr ratio of 4.1 and M/Cl ratio of 1.0 and anhydrous solid content of 28.2%. The octa salt solution has 8.8% Band I with Band III/II ratio of 1.91, while the tetra salt solution has 22.1% Band I with Band III/II ratio of 1.87. Preferred aluminum-zirconium-betaine-calcium octa-solution are those with HPLC Band I of less than 10%.

### EXAMPLE 7

Preparation of Aluminum-Strontium-Betaine and Aluminum-Zirconium-Strontium-Betaine Solutions

A basic aluminum chloride solution containing strontium was first prepared as follows:

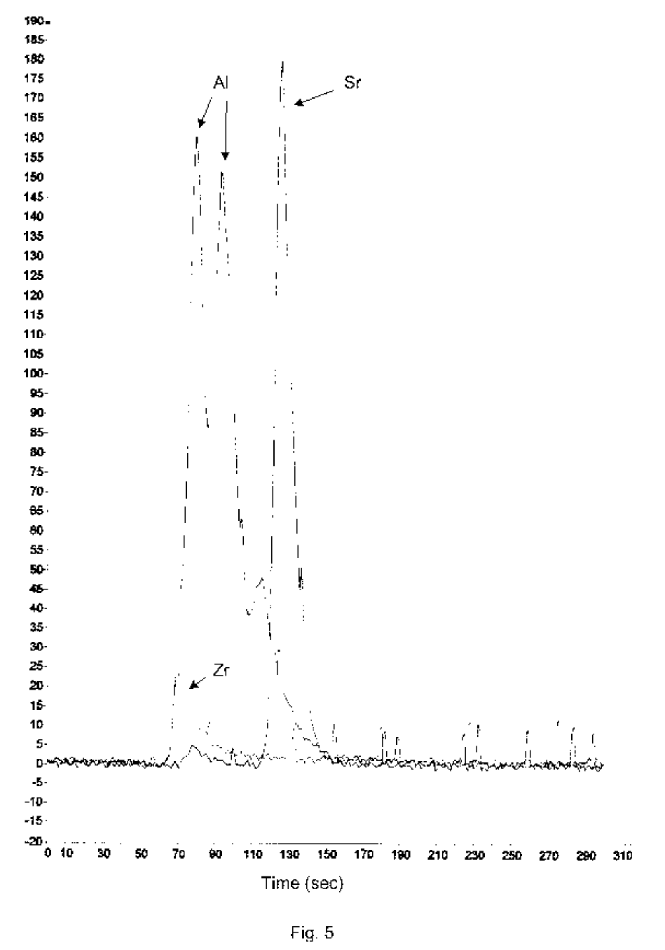

407 parts of AlCl₃ (32° Be) was mixed with 443 parts of water and heated. 62 parts of Sr(OH)₂.8H₂O (NOAH, 32% Sr) were added and the mixture was continuously heated until a clear solution was formed. 88 parts of Al powder was added and the final solution filtered to give a BAC solution containing strontium (11.01% Al, 9.18% Cl and 1.9% Sr). The BAC-strontium solution was refluxed with betaine for two hours, then mixed with ZrOCl₂ solution to form an aluminum-zirconium-betaine-strontium tetra salt-solution having a M/Cl ratio of about 1.2 and betaine/Zr ratio of about 1.8. The ICP-SEC-HPLC of the aluminum-zirconium-strontium-betaine solution is shown in FIG. 5.

### EXAMPLE 8

Preparation of Aluminum-Betaine-Calcium and Aluminum-Betaine-Strontium Solutions from Betaine HCl

458 parts of ACH solutions (10.9% Al, 7.1% Cl, Al/Cl ratio of 1.92), 40 parts of betaine HCl powders were refluxed (i) with 14 parts of CaO or (ii) or with 14 parts of Sr(OH)₂ (Aldrich, 95% purity), respectively, and the resultant solutions were filtered.

TABLE VI

| Experiment | | | | | | | HPLC |
|---|---|---|---|---|---|---|---|
| Number | % Al | % Cl | Al/Cl | % Ca | % Sr | % betaine | Band III/II |
| i | 9.33 | 8.18 | 1.49 | 0.87 | — | 6.24 | 0.85 |
| ii | 9.26 | 8.18 | 1.50 | — | 1.76 | 5.93 | 0.52 |

It will be understood that the present invention is susceptible to numerous changes and modifications as apparent to those skilled in the act. Accordingly, the present invention may be embodied in other specific forms without departing from the spirit of essential attributes of the invention disclosed herein and reference should be made to the appended claims, rather than to the foregoing specifications as indicating the scope of the invention.

What is claimed is:

1. An aqueous antiperspirant active solution comprising the reaction product of:
i. a basic aluminum chloride (BAC), having the empirical formula
Al₂(OH)₆₋ₐClₐ
where a is a number from about 1.2 to 2.0, in amounts of from about 10 to 50% by weight;
ii. betaine (trimethylglycine) in amounts of from about 2% to 15% by weight; and
iii. a metal ion selected from calcium, strontium and mixtures thereof in amounts of from about of 0.2% to 6% by weight; and
having an SEC-HPLC Band III to Band II area ratio of at least 0.5, having SEC-HPLC Band III plus Band II area of at least 70% of the total area and having SEC-HPLC Band I content of no more than 5%.

2. The antiperspirant active solution of claim 1 wherein a is from 1.4 to 1.8.

3. The antiperspirant active solution of claim 1 wherein the BAC is from about 30 to 50% by weight.

4. The antiperspirant active solution of claim 1 wherein the SEC-HPLC Band III to Band II area ratio is at least 0.7.

5. The antiperspirant active solution of claim 1 wherein betaine is present in amounts of from about 4% to 10% by weight.

6. The antiperspirant active solution of claim 1 wherein a calcium is present in amounts of from about 1% to 2.5% by weight.

7. The antiperspirant active solution of claim 1 wherein the calcium is selected from the group consisting of calcium chloride, calcium nitrate, calcium bromide, calcium citrate, calcium formate, calcium acetate, calcium ascorbate, calcium lactate, calcium carbonate, calcium sulfate, calcium hydroxide, calcium oxide and the mixture thereof.

8. The antiperspirant active solution of claim 1 wherein a strontium is present in amounts of from about 1.5% to 3% by weight.

9. The antiperspirant active solution of claim 1 wherein the strontium is selected from the group consisting of strontium chloride, strontium nitrate, strontium bromide, strontium citrate, strontium formate, strontium acetate, strontium ascorbate, strontium lactate, strontium carbonate, strontium sulfate, strontium hydroxide, strontium oxide and the mixture thereof.

10. An antiperspirant powder obtained by spray drying the solution of claim 1.

11. A method of making the antiperspirant active solution of claim 1 comprising reacting a basic aluminum salt, betaine and a salt selected from those of calcium, strontium and mixtures thereof by a process selected from
(1) heating a solution from about 10% to about 50% by weight of a basic aluminum salt of the empirical formula
Al₂(OH)₆₋ₐClₐ
wherein a is from about 1.2 to 2.0, from about 2% to 15% by weight of betaine, and salts selected from those of calcium, strontium and mixtures thereof from about of 0.5% to 15% by weight at a temperature of from 40° C. to about reflux for a period of time from about 1 hour to about 7 days;
(2) reacting Sr(OH)₂ with AlCl₃ or HCl aqueous solution, adding betaine, then reacting with Al powder from about 50° C. to about reflux for a period of time from about 1 hour to about 24 hours to afford a final solution from about 10% to about 50% by weight of a basic aluminum salt of the empirical formula
Al₂(OH)₆₋ₐClₐ
wherein a is from about 1.2 to 2.0, from about 2% to 10% by weight of betaine, and 0.5-2.5% by weight of strontium;
(3) reacting CaO with AlCl₃ or HCl aqueous solution, adding betaine, then reacting with Al powder from about 50° C. to about reflux for a period of time from about 1 hour to about 24 hours to afford a final solution from about 10% to about 50% by weight of a basic aluminum salt of the empirical formula
Al₂(OH)₆₋ₐClₐ
wherein a is from about 1.2 to 2.0, from about 2% to 10% by weight of betaine, and 0.2-1.5% by weight of calcium; and
(4) reacting Al powder with AlCl₃ or HCl aqueous solution in the presence of from about 2% to 15% by weight of betaine, and a calcium and/or strontium salt from about of 0.2% to 15% by weight from about 50° C. to about reflux for a period of time from about 1 hour to about 24 hours to afford a solution from about 10% to about 50% by weight of a basic aluminum salt of the empirical formula
Al₂(OH)₆₋ₐClₐ
wherein a is from about 1.2 to 2.0;
resulting in the said solution having an SEC-HPLC Band III to Band II area ratio of at least 0.5, having SEC-HPLC Band III plus Band II area of at least 70% of the total area and having SEC-HPLC Band I content no more than 5%.

12. The method of claim 11 wherein the aluminum to chloride atomic ratio of the resulting BAC solution is from 1.4 to 1.8.

13. The method of claim 11 wherein the resulting solution comprises from about 30% to 50% by weight BAC.

14. The method of claim 11 wherein the SEC-HPLC Band III to Band II area ratio is at least 0.7.

15. The method of claim 11 wherein betaine is present in amounts of about 4% to 10% by weight.

16. The method of claim 11 wherein the heating, in the process (1) is conducted at reflux for at least 2 hours.

17. The method of claim 11 wherein calcium in the solution of processes (1) and (4) is present in amounts of from about 1% to 2.5% by weight.

18. The method of claim 11 wherein in the processes of (1) and (4) the source of calcium is selected from the group consisting of calcium chloride, calcium nitrate, calcium bromide, calcium citrate, calcium formate, calcium acetate, calcium ascorbate, calcium lactate, calcium sulfate and the mixture thereof.

19. The method of claim 11 wherein in the solutions of (1) and (4), the strontium is present in amounts of about 1.5% to 3% by weight.

20. The method of claim 11 wherein the source of strontium in process (1) and process (4) is selected from the group consisting of strontium chloride, strontium nitrate, strontium bromide, strontium citrate, strontium formate, strontium acetate, strontium ascorbate, strontium lactate, strontium sulfate and the mixture thereof.

21. The method of claim 11 wherein 1% to 2% by weight of strontium is present in the resulting solution of process (2).

22. The method of claim 11 wherein the source of strontium in process (2) is selected from strontium carbonate, strontium hydroxide and the mixture thereof.

23. The method of claim 11 wherein 0.4% to 1% by weight of calcium is present in the resulting solution of process (3).

24. The method of claim 11 wherein the source of calcium in process (3) is selected from calcium hydroxide, calcium carbonate, calcium oxide and the mixture thereof.

25. The method of claim 11 wherein the final solution is dried to a powder.

26. An aqueous aluminum-zirconium antiperspirant active solution comprising the reaction product of:
(A) i. a basic aluminum chloride (BAC), having the empirical formula
Al₂(OH)₆₋ₐClₐ
where a is a number from about 1.2 to 2.0, in amounts of from about 10 to 50% by weight;
ii. betaine (trimethylglycine) in amounts of from about 2% to 15% by weight; and
iii. a metal ion selected from calcium, strontium and mixtures thereof in amounts of from about of 0.2% to 6% by weight;
having an SEC-HPLC Band III to Band II area ratio of at least 0.5, having SEC-HPLC Band III plus Band II area of at least 70% of the total area and having SEC-HPLC Band I content of no more than 5%;
(B) from about 15% to about 40% by weight a zirconium compound of the formula
ZrO(OH)_{b}Cl_{c}
wherein b is a numerical number from 0 to 1 and c is at least 1; and
the resulting solution having from about 1% to 10% by weight betaine (trimethylglycine) and from about of 0.2% to 5% by weight of a metal selected from calcium, strontium and mixtures thereof.

27. The antiperspirant active solution of claim 26 wherein the c for the zirconium compound is at least 1.4.

28. The antiperspirant active solution of claim 26 wherein the betaine to zirconium molar ratio is between about 0.5 and 2.

29. The antiperspirant active solution of claim 26 wherein the betaine to zirconium molar ratio is about 1.

30. The antiperspirant active solution of claim 26 wherein the M/Cl ratio is from about 0.9 to about 1.7 where M=Al+Zr.

31. The antiperspirant active solution of claim 26 wherein the M/Cl ratio is between about 1 to 1.4.

32. The antiperspirant active solution of claim 26 wherein the Al/Zr ratio is between about 2 to about 10.

33. The antiperspirant active solution of claim 26 wherein the Al—Zr composition is a tetra-salt.

34. The antiperspirant active solution of claim 26 wherein the Al—Zr composition is an octa-salt.

35. The antiperspirant active solution of claim 26 having an SEC-HPLC Band III to Band II area ratio of at least 0.7.

36. The antiperspirant active solution of claim 26 wherein the final solution is dried to a powder.

37. A method of making an aqueous aluminum—zirconium antiperspirant active solution comprising reacting:
(A) the reaction product of:
i. a basic aluminum chloride (BAC), having the empirical formula
Al₂(OH)₆₋ₐClₐ
where a is a number from about 1.2 to 2.0, in amounts of from about 10 to 50% by weight;
ii. betaine (trimethylglycine) in amounts of from about 2% to 15% by weight; and
iii. a metal calcium, ion selected from strontium and mixtures thereof in amounts of from about of 0.2% to 6% by weight;
having an SEC-HPLC Band III to Band II area ratio of at least 0.5, having SEC-HPLC Band III plus Band II area of at least 70% of the total area and having SEC-HPLC Band I content of no more than 5%;
(B) from about 15% to about 40% by weight a zirconium compound of the formula
ZrO(OH)_{b}Cl_{c}
wherein b is a numerical number from 0 to 1 and c is at least 1; and
the resulting solution having from about 1% to 10% by weight betaine (trimethylglycine) and from about of 0.2% to 5% by weight of a metal selected from calcium, strontium and mixtures thereof.

38. A topical antiperspirant composition in the form of a pump spray, roll-on, lotion, cream, or gel comprising an antiperspirant compositions according to claim 1.

39. A topical antiperspirant composition in the form of a pump spray, roll-on, lotion, cream, or gel comprising an antiperspirant compositions according to claim 26.

40. A topical antiperspirant composition in the form of an aerosol, roll-on or stick comprising an antiperspirant compositions according to claim 10.

41. A topical antiperspirant composition in the form of a stick or roll-on comprising an antiperspirant compositions according to claim 36.
